(19) United States
(12) Patent Application Publication
(10) Pub. No.: US 2017/0157300 A1
(43) Pub. Date: Jun. 8, 2017

(54) IMPLANT MADE OF BIODEGRADABLE MAGNESIUM ALLOY

(71) Applicant: BIOTRONIK AG, Buelach (CH)

(72) Inventor: Bodo Gerold, Zellingen (DE)

(21) Appl. No.: 15/440,921

(22) Filed: Feb. 23, 2017

Related U.S. Application Data
(63) Continuation of application No. 13/635,039, filed on Sep. 14, 2012.
(60) Provisional application No. 61/317,296, filed on Mar. 25, 2010.

(51) Int. Cl. A61L 31/02; C22C 23/06; A61F 2/82; A61L 31/14

(52) U.S. Cl. CPC A61L 31/022; A61L 31/148; C22C 23/06; A61F 2/82; A61L 2400/18; A61F 2210/0004

(57) ABSTRACT

The present invention relates to implants made of a biodegradable magnesium alloy. The inventive implant is made in total or in parts of a biodegradable magnesium alloy with particular composition and impurities such that it has improved processability during manufacture especially in new highly sophisticated techniques like micro-extrusion and, if applicable, improved mechanical properties of the material, such as strength, ductility and strain hardening. In particular, when the implant is a stent, scaffolding strength of the final device as well as the tube drawing properties are favourable.

### REFERENCE TO RELATED APPLICATION AND PRIORITY CALIM

This application is a continuation of and claims priority under 35 U.S.C. §120 from application Ser. No. 13/635,039, filed Sep. 14, 2012 (incorporated by reference herein), which application was the U.S. National Phase under 35 U.S.C. §371 of International Application No. PCT/EP2011/054448, filed Mar. 23, 2011, which claims priority to U.S. Provisional Application No. 61/317,296, filed Mar. 25, 2010.

### FIELD OF THE INVENTION

The present invention relates to implants made of a biodegradable magnesium alloy.

### BACKGROUND OF THE INVENTION

Medical implants for greatly varying uses are known in the art. A shared goal in the implementation of modern medical implants is high biocompatibility, i.e., a high degree of tissue compatibility of the medical product inserted into the body. Frequently, only a temporary presence of the implant in the body is necessary to fulfil the medical purpose. Implants made of materials which do not degrade in the body are often to be removed again, because rejection reactions of the body may occur in the long term even with highly biocompatible permanent materials.

One approach for avoiding additional surgical intervention is to form the implant entirely or in major parts from a biodegradable (or biocorrodible) material. The term biodegradation as used herewith is understood as the sum of microbial procedures or processes solely caused by the presence of bodily media, which result in a gradual degradation of the structure comprising the material. At a specific time, the implant, or at least the part of the implant which comprises the biodegradable material, loses its mechanical integrity. The degradation products are mainly resorbed by the body, although small residues are in general tolerable. Biodegradable materials have been developed, inter alia, on the basis of polymers of a synthetic nature or natural origin. Because of the material properties, but particularly also because of the degradation products of the synthetic polymers, the use of biodegradable polymers is still significantly limited. Thus, for example, orthopedic implants must frequently withstand high mechanical strains, and vascular implants, e.g., stents, must meet very special requirements for modulus of elasticity, brittleness, and formability depending on their design.

One promising attempted achievement provides the use of biodegradable metal alloys. For example, it is suggested in German Patent Application No. 197 31 021 A1 to form medical implants from a metallic material whose main component is to be selected from the group of alkali metals, alkaline earth metals, iron, zinc, and aluminium. Alloys based on magnesium, iron, and zinc are described as especially suitable. Secondary components of the alloys may be manganese, cobalt, nickel, chromium, copper, cadmium, lead, tin, thorium, zirconium, silver, gold, palladium, platinum, silicon, calcium, lithium, aluminium, zinc, and iron.

The use of a biodegradable magnesium alloy having a proportion of magnesium greater than 90% by weight, yttrium 3.7-5.5% by weight, rare earth metals 1.5-4.4% by weight, and the remainder less than 1% by weight is known from European Patent 1 419 793 B1. The material disclosed therein is in particular suitable for producing stents.

Another intravascular implant is described in European Patent Application 1 842 507 A1, wherein the implant is made of a magnesium alloy including gadolinium and the magnesium alloy is being free of yttrium.

Stents made of a biodegradable magnesium alloy are already in clinical trials. In particular, the yttrium (W) and rare earth elements (E) containing magnesium alloy ELEKTRON WE43 (U.S. Pat. No. 4,401,621) of Magnesium Elektron, UK, has been investigated, wherein a content of yttrium is about 4% by weight and a content of rare earth metals (RE) is about 3% by weight. The following abbreviations are often used: RE=rare earth elements, LRE=light rare earth elements (La—Pm) and HRE=heavy rare earth elements (Sm—Lu). However, it was found that the alloys respond to thermo-mechanical treatments. Although these types of WE alloys originally were designed for high temperature applications where high creep strength was required, it has now been found that dramatic changes in the microstructure occurred during processing with repetitive deformation and heat treatment cycles. These changes in the microstructure are responsible for high scrap rates during production and inhomogeneous properties of seamless tubes and therefore in the final product. As a consequence, mechanical properties are harmfully affected. Especially, the tensile properties of drawn tubes in the process of manufacturing stents are deteriorated and fractures appear during processing. In addition, a large scatter of the mechanical properties especially the elongation at fracture (early fractures of the tubes below yield strength during tensile testing) was found in the final tube. Finally, the in vivo degradation of the stent is too fast and too inhomogeneous, and therefore the biocompatibility may be worse due to the inflammation process caused by a tissue overload of the degradation products.

The use of mixtures of light rare earth elements (LRE; La, Ce, Pr, Nd) and heavy rare earth metals (HRE; elements of the periodic table: Sm, Eu, Gd, Tb, Dy, Ho, Er, Tm, Yb and Lu) in commercially available magnesium alloys such as WE43 rather than pure alloying elements reduced the costs, and it has been demonstrated that the formation of additional precipitates of these elements beside the main precipitates based on Y and Nd further enhance the high temperature strength of the material [King et al, 59^{th} Annual World Magnesium Conference, 2005, p. 15ff]. It could therefore be postulated that the HRE containing precipitates are more stable against growth at higher temperatures because of the significantly slower diffusion rate of these elements compared to Y and Nd. Therefore, they contribute substantially to the high temperature strength of WE alloys (particle hardening effect).

However, it now has been found that these HRE precipitates are causing problems when the material is used in biomedical applications, such as vascular implants (e.g. stents) or in orthopaedic implants. The HRE intermetallic particles can adversely affect the thermomechanical processability of alloys. For example, manufacturing vascular prostheses like stents made of metallic materials usually starts from drawn seamless tubes made of the material. The production of such seamless tubes is usually an alternating process of cold deformation by drawing and subsequent thermal treatments to restore the deformability and ductility, respectively. During the mechanical deformation steps, intermetallic particles cause problems because they usually have significantly higher hardness than the surrounding matrix. This leads to crack formation in the vicinity of the particles and therefore to defects in the (semi-finished) parts which reduces their usability in terms of further processing by drawing and also as final parts for production of stents.

Surprisingly, it now has been found that precipitation still happens to occur although the temperature regime is high enough that one would expect dissolution of all existing particles. This indicates that the intermetallic phases predominantly formed with LRE cannot be dissolved during usual recrystallization heat treatment (300 to 525° C.) of the specific alloys. As a consequence, the ductility for further deformation processes or service cannot be restored sufficiently.

As mentioned above, magnesium has many advantages for biomedical applications, for example biodegradable inserts like stents, screws/plates for bone repair and surgical suture materials. For many applications however, the time for degradation and failure of the magnesium repair device is too soon and can develop too much gas evolution (H₂) during the corrosion process. Additionally, the failure of stressed magnesium devices can occur due to Environmentally Assisted Cracking (EAC). EAC, which is also referred to as Stress Corrosion Cracking (SCC) or Corrosion Fatigue (CF), is a phenomenon which can result in catastrophic failure of a material. This failure often occurs below the Yield Strength (YS). The requirement for EAC to occur is three fold: namely mechanical loading, susceptible material, and a suitable environment.

ECSS (European Cooperation for Space Standardisation), quantifies the susceptibility of various metallic alloys by use of an industry recognised test, employing aqueous NaCl solution. ECSS-Q-70-36 report ranks the susceptibility of several Magnesium alloys, including Mg—Y—Nd—HRE-Zr alloy WE54. This reference classifies materials as having high, moderate, or low resistance to SCC. WE54 is classed as “low resistance to SCC” (ie poor performance). For Biomedical applications, stresses are imposed on the materials, and the in vivo environment (e.g. blood) is known to be the most corrosive. As for the ECSS tests, SBF (simulated body fluid), which is widely used for in vitro testing, includes NaCl. Tests described in this patent application suggest that EAC performance of the Mg—Y—Nd—HRE-Zr alloy system can be improved by selective use of HRE additions. This offers a significant benefit for biomedical implants, where premature failure could have catastrophic results. For example, Atrens (Overview of stress corrosion cracking of magnesium alloys—8^{th} International conference on Magnesium alloys—DGM 2009) relates to the potential use of stents in heart surgery, whereby fracture due to SCC would probably be fatal. The consequence of premature failure may include re-intervention, patient trauma, etc. The alloys used must still be formable and show sufficient strength.

### SUMMARY OF THE INVENTION

An aim of this invention is to overcome, or to at least lower, one or more of the above mentioned problems. There is a demand for a biodegradable Mg alloy having improved processability, especially in new highly sophisticated techniques like micro-extrusion and, if applicable, improved mechanical properties of the material, such as strength, ductility and strain hardening. In some embodiments, when the implant is a stent, the scaffolding strength of the final device as well as the tube drawing properties of the material should be improved.

In some embodiments, a further aspect of the invention may be to enhance the corrosion resistance of the material, and more specifically, to slow the degradation, to fasten the formation of a protective conversion layer, and to lessen the hydrogen evolution. In the case of a stent, enhancing the corrosion resistance will lengthen the time wherein the implant can provide sufficient scaffolding ability in vivo.

In some embodiments, another aspect of the invention may be to enhance the biocompatibility of the material by avoiding toxic components in the alloy or the corrosion products.

### BRIEF DESCRIPTION OF THE DRAWINGS

FIGS. 1-7 show microstructures of samples; and

FIG. 8 shows an example of secondary cracking caused by EAC in SBF solution.

FIG. 9 shows the evolution of the relative collapse pressure of stents during corrosion fatigue testing.

FIG. 10 shows the evolution of the relative load bearing cross section of stents during corrosion fatigue testing.

### DETAILED DESCRIPTION OF THE INVENTION

One or more of the above mentioned aspects can be achieved by the implant of the present invention. The inventive implant is made in total, or in parts, of a biodegradable magnesium alloy comprising or consisting essentially of:
Y: 0-10.0% by weight
Nd: 0-4.5% by weight
Gd: 0-9.0% by weight
Dy: 0-8.0% by weight
Ho: 0-19.0% by weight
Er: 0-23.0% by weight
Lu: 0-25.0% by weight
Tm: 0-21.0% by weight
Tb: 0-21.0% by weight
Zr: 0.1-1.5% by weight
Ca: 0-2.0% by weight
Zn: 0-1.5% by weight
In: 0-12.0% by weight
Sc: 0-15.0% by weight
incidental impurities up to a total of 0.3% by weight
the balance being magnesium and under the condition that
a) a total content of Ho, Er, Lu, Tb and Tm is more than 5.5% by weight;
b) a total content of Y, Nd and Gd is more than 2% by weight; and
c) a total content of all alloy compounds except magnesium is more than 8.5% by weight.

In alternative, the inventive implant is made in total, or in parts, of a biodegradable magnesium alloy consisting of:
Y: 0-10.0% by weight
Nd: 0-4.5% by weight
Gd: 0-9.0% by weight
Dy: 0-8.0% by weight
Ho: 0-19.0% by weight
Er: 0-23.0% by weight
Lu: 0-25.0% by weight
Tm: 0-21.0% by weight
Tb: 0-21.0% by weight
Zr: 0.1-1.5% by weight
Ca: 0-2.0% by weight
Zn: 0-1.5% by weight
In: 0-12.0% by weight
Sc: 0-15.0% by weight
incidental impurities up to a total of 0.3% by weight
the balance being magnesium and under the condition that
a) a total content of Ho, Er, Lu, Tb and Tm is more than 5.5% by weight;
b) a total content of Y, Nd and Gd is more than 2% by weight; and
c) a total content of all alloy compounds except magnesium is more than 8.5% by weight.

The use of the inventive Mg alloy for manufacturing an implant causes an improvement in processability, and an increase in corrosion resistance and biocompatibility, compared to conventional magnesium alloys, especially WE alloys such as WE43 or WE54.

TABLE 1
Solid solubility of various LRE and HRE in magnesium

| Atomic number | Element | RT | 300° C. | 400° C. | 500° C. |
|---|---|---|---|---|---|
| 71 | Lu | 10-12 | 19.5 | 25 | 35 |
| 70 | Yb | ca. 0 | 0.5 | 1.5 | 3.3 |
| 69 | Tm | 10-12 | 17.6 | 21.7 | 27.5 |
| 68 | Er | 10-12 | 18.5 | 23 | 28.3 |
| 67 | Ho | 8-10 | 15.4 | 19.4 | 24.2 |
| 66 | Dy | ca. 5 | 14 | 17.8 | 22.5 |
| 65 | Tb | 1-2 | 12.2 | 16.7 | 21.0 |
| 64 | Gd | ca. 0 | 3.8 | 11.5 | 19.2 |
| 63 | Eu | 0 | 0 | 0 | 0 |
| 62 | Sm | ca. 0 | 0.8 | 1.8 | 4.3 |
| 61 | Pm | — | — | — | — |
| 60 | Nd | ca. 0 | 0.16 | 0.7 | 2.2 |
| 59 | Pr | ca. 0 | 0.05 | 0.2 | 0.6 |
| 58 | Ce | ca. 0 | 0.06 | 0.08 | 0.26 |
| 57 | La | ca. 0 | 0.01 | 0.01 | 0.03 |
| 39 | Y | 1-2 | 4.2 | 6.5 | 10.0 |
| 21 | Sc | ca. 12 | 12.8 | 15.7 | 18.8 |

The solubility of RE in magnesium varies considerably; see Table 1. It may be expected from one skilled in the art, that the volume of coarse particles present would be primarily related to the Nd content, due to the low solid solubility of this element. Therefore, the amount of RE addition may be expected to affect the amount of retained clusters and particles present in the microstructure.

Examination of the microstructure of some of the inventive magnesium alloys and conventional WE43 revealed that for specific compositions there were significantly less and smaller precipitates in the inventive magnesium alloy than in WE43.

In other words, the selection of the type of RE present in the Mg alloy has surprisingly led to an improvement in the formability characteristics, although the total amount of RE is significantly increased. It is proposed that this improvement is achieved by a reduction in the hard particles (precipitates).

The content of Y in the Mg alloy is typically 0-10.0% by weight. Preferably, the content of Y in the Mg alloy is 1.0-6.0% by weight; and the most preferred is 3.0-4.0% by weight. Keeping the content of Y within the ranges ensures that the consistency of the properties, e.g. scatter during tensile testing, is maintained. Further, strength and corrosion behaviour is improved. When the content of Y is above 10.0% by weight, the ductility of the alloy is deteriorated.

The content of Nd in the Mg alloy is typically 0-4.5% by weight, and preferably 0.05-2.5% by weight. When the content of Nd is above 4.5% by weight, the ductility of the alloy is deteriorated due to a limited solubility of Nd in Mg.

The content of Gd in the Mg alloy is typically 0-9.0% by weight, and preferably 0-4.0% by weight. Gd can reduce the degradation of the alloy in SBF tests and improve its EAC behaviour. Levels of Gd approaching the solubility limit in a given alloy reduce ductility.

A total content of Y, Nd and Gd in the Mg alloy is typically more than 2.0% by weight, and preferably more than 3.0% by weight.

The content of Dy in the Mg alloy is typically 0-8.0% by weight, preferably 0-6.0% by weight, and the most preferred is 0-4.0% by weight.

The content of Ho in the Mg alloy is typically 0-19.0% by weight, preferably 4.0-15.0% by weight, and the most preferred is 6.0-14.0% by weight. Ho can reduce the degradation of the alloy in SBF and increases strength.

The content of Er in the Mg alloy is typically 0-23.0% by weight, preferably 4.0-15.0% by weight, and the most preferred is 6.0-14.0% by weight. Er can reduce the degradation of the alloy in SBF tests and improve its EAC behaviour and strength.

The content of Lu in the Mg alloy is typically 0-25.0% by weight, preferably 4.0-15.0% by weight, and the most preferred is 6.0-14.0% by weight. Lu can reduce the degradation of the alloy in SBF tests and improve its EAC behaviour and strength.

The content of Tm and/or Tb in the Mg alloy is typically 0-21.0% by weight, preferably 4.0-15.0% by weight, and the most preferred is 6.0-12.0% by weight. For Tb and Tm, the same effect on degradation of the alloy and improvement of the EAC behaviour and strength is expected.

A total content of Ho, Er, Lu, Tb and Tm in the Mg alloy is typically more than 5.5% by weight. Preferably, the total content of Ho, Er, Lu, Tb and Tm in the Mg alloy is 6.5-25.0% by weight, and the most preferred is 7.0-15.0% by weight. Preferably, the total content includes Dy as additional element.

In addition, the content of Zr in the Mg alloy is typically 0.1-1.5% by weight, preferably 0.2-0.6% by weight, and the most preferred is 0.2-0.4% by weight. For magnesium-zirconium alloys, zirconium has a significant benefit of reducing the grain size of the magnesium alloys, especially of the pre-extruded material, which improves the ductility of the alloy. Further, Zr removes contaminants from the melt.

The content of Ca in the Mg alloy is typically 0-2.0% by weight, preferably 0-1.0% by weight, and the most preferred is 0.1-0.8% by weight. Ca has a significant benefit of reducing the grain size of magnesium alloys.

The content of Zn in the Mg alloy is typically 0-1.5% by weight, preferably 0-0.5% by weight, and the most preferred is 0.1-0.3% by weight. Zn can contribute to precipitation and can also affect general corrosion.

The content of In in the Mg alloy is typically 0-12.0% by weight, preferably 0-2.5% by weight, and the most preferred is 0.0-0.8% by weight. In has a benefit of improving the corrosion performance of magnesium alloys. Additionally In has a benefit of reducing the grain size of magnesium alloy.

A total content of In, Zr, Ca and Zn in the Mg alloy is typically in the range of 0.2-2.0% by weight, and preferably 0.2-0.8% by weight.

The content of Sc in the Mg alloy is typically 0-15% by weight. Sc can have a positive effect on corrosion resistance.

The total content of impurities in the alloy should be less than 0.3% by weight, and more preferred less that 0.2% by weight. In particular, the following maximum impurity levels should be preserved:

[symbol] Fe, Si, Cu, Mn, and Ag each less than 0.05% by weight

[symbol] Ni less than 0.006% by weight

[symbol] La, Ce, Pr, Sm, Eu and Yb less than 0.15% by weight, preferably less than 0.1% by weight

For the purposes of the present invention, alloys are referred to as biodegradable in which degradation occurs in a physiological environment, which finally results in the entire implant or the part of the implant formed by the material losing its mechanical integrity. Artificial plasma, has been previously described according to EN ISO 10993-15:2000 for biodegradation assays (composition NaCl 6.8 g/l. CaCl₂ 0.2 g/l. KCl_{0.4} g/l. MgSO₄ 0.1 g/l. is NaHCO₃ 2.2 g/l. Na₂HPO₄ 0.126 g/l. NaH₂PO₄ 0.026 g/l), is used as a testing medium for testing the corrosion behaviour of an alloy coming into consideration. For this purpose, a sample of the alloy to be assayed is stored in a closed sample container with a defined quantity of the testing medium at 37° C. At time intervals tailored to the corrosion behaviour to be expected, a few hours up to multiple months, the sample is removed and examfined for corrosion traces in a known way.

Implants are devices introduced into the human body via a surgical method and comprise fasteners for bones, such as screws, plates, or nails, surgical suture material, intestinal clamps, vascular clips, prostheses in the area of the hard and soft tissue, and anchoring elements for electrodes, in particular, of pacemakers or defibrillators. The implant is preferably a stent. Stents of typical construction have filigree support structures made of metallic struts which are initially provided in an unexpanded state for introduction into the body and are then widened into an expanded state at the location of application.

Vascular implants, especially stents, are preferably to be designed in regard to the alloys used in such a way that a mechanical integrity of the implant is maintained for 2 through 20 weeks. Implants as an occluder are preferably to be designed in regard to biodegradability in such a way that the mechanical integrity of the implant is maintained for 6 through 12 months. Orthopedic implants for osteosynthesis are preferably to be designed in regard to the magnesium alloy in such a way that the mechanical integrity of the implant is maintained for 6 through 36 months.

The present disclosure is explained in greater detail in the following on the basis of exemplary embodiments and the associated drawings.

Several melts with different alloy compositions were melted cast, and extruded, and subsequently subject to different investigation with the emphasis on the microstructure (grain size, size, fraction and composition of precipitates), the respective thermo-mechanical properties (tensile properties), and the corrosion behaviour with and without a superimposed mechanical load. In addition, biocompatibility tests were carried out. In general, melts were carried out according to the following casting technique:

High-purity starting materials (≧99.9%) were melted in steel crucibles under a protective gas (CO₂/2% SF₆). The temperature was raised to 760° C. to 800° C. before the melt was homogenized by stirring. The melt was cast to form bars with a nominal diameter of 120 mm and a length of 300 mm. Next the bars were machined to a nominal diameter of 75 mm with a length of 150 mm to 250 mm and homogenized for 4-8 hours at approximately 525° C.

The material was then heated to 350-500° C. and extruded with the help of a hydraulic press. The resulting round rods had a diameter in the range of 6 mm to 16 mm, mostly 9.5-12.7 mm. For the following investigations, pieces from the start and end of an extrusion 30 cm long were usually removed.

Table 2 summarises the chemical compositions, corrosion rates and tensile properties of exemplary Mg alloys. MI0007, MI0034 and DF4619 are comparative examples of WE43 within AMS4427 chemical specification used as reference material. Each time, melts were produced to generate tensile data and for metallography.

TABLE 2

| Chemical Analysis | | | | | | | | | | |
|---|---|---|---|---|---|---|---|---|---|---|
| Y | Nd | Zr | Gd | Dy | Yb | Er | Sm | La | Ce | Pr |

| | |
|---|---|
| ID | % by weight |

Reference alloys

| | | | | | | | | | | |
|---|---|---|---|---|---|---|---|---|---|---|
| MI0034 | 3.8 | 2.3 | 0.54 | 0.44 | 0.47 | 0 | 0.01 | 0.01 | 0.00 | — |
| MI0007 | 4 | 2.2 | 0.57 | 0.46 | 0.46 | 0 | — | — | 0.00 | — |
| DF9375 | 3.9 | 2.15 | 0.51 | 0.28 | 0.36 | 0.03 | 0.02 | 0.02 | 0.06 | — |
| DF4619a | 3.9 | 2.2 | 0.56 | 0.28 | 0.30 | 0.03 | 0.09 | 0.03 | 0.00 | 0.00 |

Effect of Er levels

| | | | | | | | | | | | |
|---|---|---|---|---|---|---|---|---|---|---|---|
| DF9546 | 4.00 | 0.00 | 0.63 | 0.00 | 0.00 | 0.00 | 6.61 | 0.00 | 0.00 | 0.00 | 0.00 |
| DF9561 | 4.00 | 2.20 | 0.61 | 0.00 | 0.00 | 0.00 | 7.14 | 0.02 | 0.00 | 0.00 | 0.00 |
| MI0023 | 3.90 | 2.30 | 0.57 | 0.48 | 0.54 | 0.00 | 7.35 | 0.02 | 0.00 | 0.01 | 0.00 |
| MI0029 | 4.20 | 2.20 | 0.59 | 0.00 | 0.02 | 0.00 | 7.65 | 0.02 | 0.00 | 0.01 | 0.00 |
| MI0030 | 4.10 | 2.30 | 0.62 | 0.00 | 0.03 | 0.00 | 12.72 | 0.03 | 0.01 | 0.02 | 0.01 |
| MI0036 | 3.60 | 0.03 | 0.83 | 0.00 | 0.00 | 0.00 | 14.00 | 0.01 | 0.00 | 0.01 | 0.00 |
| MI0037 | 3.90 | 0.04 | 0.80 | 0.00 | 0.00 | 0.00 | 18.00 | 0.03 | 0.00 | 0.03 | 0.00 |

Effect of Y, Nd levels

| | | | | | | | | | | | |
|---|---|---|---|---|---|---|---|---|---|---|---|
| MI0030 | 4.10 | 2.30 | 0.62 | 0.00 | 0.03 | 0.00 | 12.72 | 0.03 | 0.01 | 0.02 | 0.01 |
| MI0031 | 3.90 | 0.00 | 0.74 | 0.00 | 0.02 | 0.00 | 12.69 | 0.02 | 0.00 | 0.02 | 0.00 |
| MI0041 | 0.07 | 0.03 | 0.90 | 0.00 | 0.00 | 0.00 | 12.50 | 0.01 | 0.00 | 0.01 | 0.00 |
| MI0042 | 2.1 | 0.21 | 0.80 | 0.00 | 0.01 | 0.00 | 12.50 | 0.01 | 0.00 | 0.01 | 0.00 |
| MI0043 | 2.2 | 1.12 | 0.75 | 0.00 | 0.00 | 0.00 | 12.50 | 0.01 | 0.00 | 0.01 | 0.00 |
| MI0044 | 1.9 | 1.93 | 0.70 | 0.00 | 0.01 | 0.00 | 12.50 | 0.01 | 0.00 | 0.00 | 0.00 |

Effect of Ho, Lu

| | | | | | | | | | | | |
|---|---|---|---|---|---|---|---|---|---|---|---|
| MI0023 | 3.90 | 2.30 | 0.57 | 0.48 | 0.54 | 0.00 | 7.35 | 0.02 | 0.00 | 0.01 | 0.00 |
| MI0045 | 3.7 | 2.07 | 0.73 | 0.43 | 0.29 | 0.00 | 0.03 | 0.06 | 0.00 | 0.03 | 0.00 |
| MI0046 | 4.2 | 2.06 | 0.81 | 0.25 | 0.29 | 0 | 0.04 | 0.008 | 0 | 0 | 0.003 |

Effect of Gd & Er

| | | | | | | | | | | | |
|---|---|---|---|---|---|---|---|---|---|---|---|
| MI0026 | 3.90 | 2.30 | 0.48 | 3.60 | 0.48 | 0.00 | 7.70 | 0.05 | 0.00 | 0.05 | 0.04 |

Additional Examples

| MI0024 | 4.00 | 2.40 | 0.60 | 0.00 | 0.00 | 0.00 | 1.74 | 0.00 | 0.00 | 0.00 | 0.00 |
|---|---|---|---|---|---|---|---|---|---|---|---|
| | | | | | | | | | Corrosion Properties | | |
| | | | | | | Tensile Properties | | | Salt | Immersion | % of WE43 |
| | | | | | | 0.2% | | | fog in | in | Reference |
| | Ho | Lu | Al | Fe | TRE ¹ | YS | UTS | Elong | NaCl | SBF | alloy |

| | | | | | | | |
|---|---|---|---|---|---|---|---|
| ID | % by weight | MPa | MPa | % | mpy | mpy | % |

Reference alloys

| | | | | | | | | | |
|---|---|---|---|---|---|---|---|---|---|
| MI0034 | 0.01 | 0.002 | 0.9 | 210 | 290 | 26 | 12 | 775 | 100 |
| MI0007 | 0.01 | 0.002 | 0.9 | 210 | 291 | 26 | 14 | 835 | |
| DF9375 | 0.01 | 0.002 | 0.8 | 199 | 276 | 26 | 43 | 960 | |
| DF4619a | 0.002 | 0.002 | 0.7 | 209 | 298 | 19 | 56 | 1325 | |

Effect of Er levels

| | | | | | | | | | |
|---|---|---|---|---|---|---|---|---|---|
| DF9546 | 0.001 | 0.002 | 6.6 | 195 | 283 | 24 | 12 | 530 | 40 |
| DF9561 | 0.01 | 0.003 | 7.2 | 218 | 309 | 23 | 25 | 614 | 49 |
| MI0023 | 0.01 | 0.002 | 8.4 | 276 | 348 | 14 | 18 | 206 | 40 |
| MI0029 | 0.01 | 0.003 | 7.7 | 246 | 322 | 18 | 61 | 73.2 | 48 |
| MI0030 | 0.01 | 0.004 | 12.8 | 302 | 370 | 10 | 74 | 36.1 | 23 |
| MI0036 | 0.01 | 0.004 | 14.0 | 265 | 341 | 19 | 432 | 48 | 8 |
| MI0037 | 0.01 | 0.004 | 18.1 | 302 | 353 | 2 | 1800 | 142 | 25 |

Effect of Y, Nd levels

| | | | | | | | | | |
|---|---|---|---|---|---|---|---|---|---|
| MI0030 | 0.01 | 0.004 | 12.8 | 302 | 370 | 10 | 74 | 36.1 | 23 |
| MI0031 | 0.01 | 0.003 | 12.8 | 258 | 337 | 19 | NA | 86.8 | 14 |
| MI0041 | 0.01 | 0.003 | 12.5 | 159 | 240 | 24 | 42 | 252 | 65 |
| MI0042 | 0.01 | 0.003 | 12.5 | 218 | 298 | 22 | 1588 | 104 | 27 |
| MI0043 | 0.01 | 0.003 | 12.5 | 248 | 318 | 15 | 425 | 106 | 28 |
| MI0044 | 0.007 | 0.003 | 12.5 | 248 | 323 | 18 | 1976 | 278 | 72 |

Effect of Ho, Lu

| | | | | | | | | | | | |
|---|---|---|---|---|---|---|---|---|---|---|---|
| MI0023 | | | 0.01 | 0.002 | 8.4 | 276 | 348 | 14 | 18 | 206 | 40 |
| MI0045 | 8 | | 0.008 | 0.003 | 8.8 | 245 | 327 | 23 | 45 | 100 | 26 |
| MI0046 | | 8 | 0.001 | 0.003 | 8.6 | 228 | 307 | 24 | 22 | 66 | 17 |

Effect of Gd & Er

| | | | | | | | | | |
|---|---|---|---|---|---|---|---|---|---|
| MI0026 | 0.01 | 0.002 | 11.9 | 299 | 369 | 11 | 9 | 276 | 54 |

Additional Examples

| | | | | | | | | | |
|---|---|---|---|---|---|---|---|---|---|
| MI0024 | 0.01 | 0.002 | 1.7 | 217 | 294 | 23 | 15 | 477 | 92 |

Mechanical Properties and Metallurgical Description

To determine the mechanical properties, standardized tension tests of the bulk materials were performed and analyzed using several samples of a melt in each case. The 0.2% yield tensile strength (YTS), the ultimate tensile strength (UTS), and elongation at fracture (A) were determined as characteristic data. The yield strength YS of a material is defined as the stress at which material strain changes from elastic deformation to plastic deformation, causing it to deform permanently. The ultimate tensile strength UTS is defined as the maximum stress a material can withstand before break.

In addition, tensile tests were also performed with extruded tubes and drawn tubes as references. The typical extruded tubes have a typical length of not less than 30 mm, a diameter of ca. 2 mm and a wall thickness between 50 and 400 μm. They are processed by a hot micro extrusion process at temperatures between 200° C. and 480° C. and extrusion speeds of 0.1 mm/s to 21 mm/s.

For the metallographic examination of the as extruded condition, the materials were melted, cast, homogenized, cut to billets and extruded to bars. Then samples were cut, embedded in epoxy resin, ground, polished to a mirror like finish and etched according to stand and metallographic techniques [G Petzow, Metallographisches, keramographisches and plastographisches Atzen, Borntraeger 2006].

Discussion of Bulk Material Mechanical Properties

Table 2 summarizes the chemical composition, mechanical (tensile test), and corrosion (salt fog in NaCl and immersion in SBF) properties of Mg alloys. As can be seen form the data of Table 2, the inventive changes in the composition of the alloys affect the tensile properties compared to the reference in terms of strength and ductility.

One skilled in the art may expect increasing strength and decreasing ductility with increasing amount of appropriate alloying elements. This can actually be observed in Table 2.

For example, increasing Er content in the approximate range >2% to 8% increases strength and maintains ductility. For higher values of Er, ductility is seen to decline. A reduction in other elements, for example Nd, can compensate for the Er addition, minimizing the effect of Er on ductility and allowing higher amounts of Er to be added. For example, MI0036 shows an example of good ductility with 14% Er addition.

Small changes in other RE additions may affect ductility—for example Gd and Dy (MI0023 vs. DF9561).

Similar effects may be seen for major additions of other REs, for example Ho, Lu, and Gd; however, different threshold values compared with Er are suggested before ductility falls.

It is expected from the data that higher levels than the 8% examples shown of, for example Ho and Lu, could be employed without the loss of significant ductility, and probably to higher values than the equivalent Er containing examples.

As can be seen from the data of Table 2, the inventive changes of the amount of Y and Nd in the composition of the alloys basically effects strength, ductility, and tolerance of some other REs.

Combinations of REs (for example Gd and Er) are not always synergistic; however, certain combinations are expected to be beneficial.

TABLE 3
Mechanical properties of bulk material and micro-extruded tubes

| | | | | extruded bulk + | | |
|---|---|---|---|---|---|---|
| | extruded bulk | | | micro-extruded | | |
| | YTS | UTS | A | YTS | UTS | A |
| ID | in MPa | In MPa | in % | in MPa | in MPa | in % |
| DF9375 | 199 | 276 | 26 | 164 | 250 | 20 |
| Reference | | | | | | |
| DF9546 | 195 | 283 | 24 | 173 | 261 | 29 |
| DF9561 | 218 | 309 | 23 | 189 | 316 | 26 |
| MI0023 | 276 | 348 | 14 | 227 | 364 | 20 |
| MI0012 | 299 | 369 | 11 | 206 | 361 | 16 |

For applications, the extruded bulk material is often processed further to achieve a product. This processing can include drawing, rolling and bending steps, and other advanced processing techniques. It has now been discovered that, surprisingly, alloys of the invention show an improvement during such subsequent processing steps, for example micro extrusion.

The comparison of the tensile properties between typical inventive alloys and the reference material before and after micro-extrusion clearly indicate that the inventive alloys are more susceptible to thermo-mechanical treatments, in particular micro-extrusion.

The inventive alloy show a significant drop of 10-30% in yield strength for all tested inventive alloys, minor changes of about plus or minus 10% in ultimate strength depending on the inventive alloy, and a significant rise of 10-50% in ductility for all tested inventive alloys.

All of these effects are desirable because the effect of lower YS combined with more or less the same UTS leads to a significantly lower (minus 5-30%) yield-to-tensile strength ratio of less than 0.6. Together with the higher ductility, this is beneficial, for example, in terms of developing stent designs with, for example, homogenous opening behaviour and higher radial strength. In contrast, the reference material exhibits about 20% drop in yield strength, about 10% drop in ultimate strength, and about 20% drop in ductility.

Microstructure

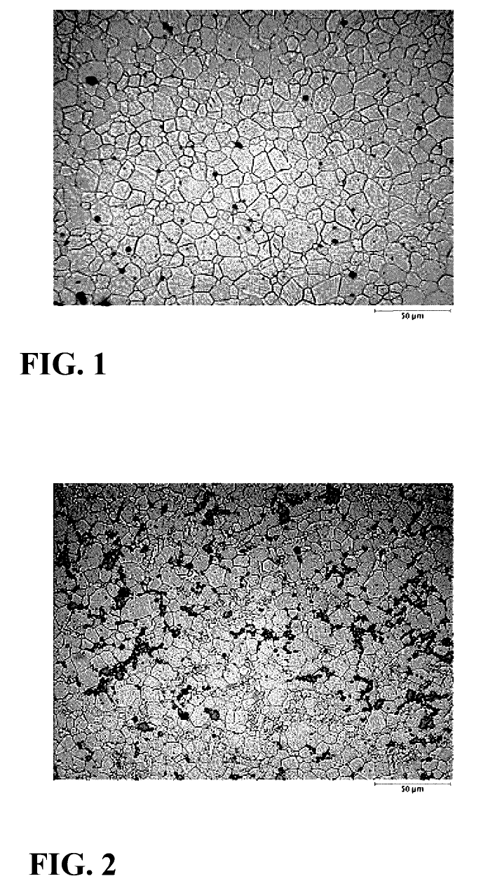

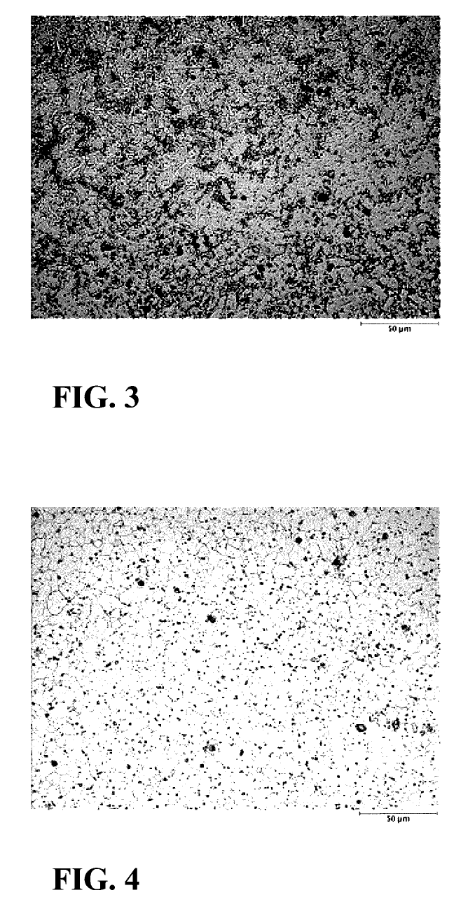

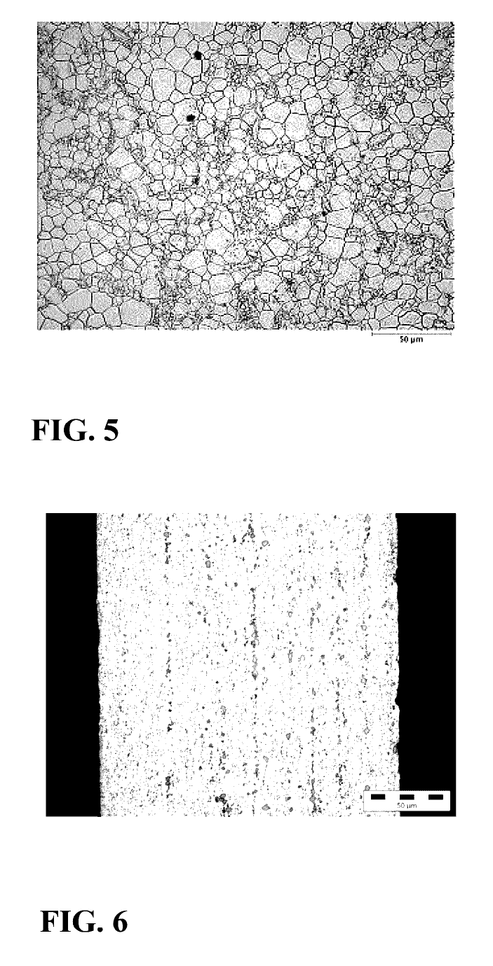

FIGS. 1 through 5 show the microstructures of exemplary samples (FIG. 1: MI0031/FIG. 2: MI0030/FIG. 3: MI0037/FIG. 4: MI0029/FIG. 5: MI0046) after extrusion. They provide an insight into the effect of the alloy composition upon the strength and ductility of some of the alloy examples. A microstructure which is free of large particles and clusters (“clean microstructure”) can offer the advantage of improved ductility if the clusters/particles are brittle.

FIG. 1 is a comparatively “clean microstructure” despite a 12.7% addition of Er, and the ductility is good (19%).

FIG. 2 shows the effect of adding Nd to the alloy of FIG. 1. The microstructure has more clusters, and the ductility falls (10%). It will, however, be noticed that the alloy of FIG. 1 possess higher tensile properties.

FIG. 3 contains a higher level of Er (18%) than the alloy of FIG. 1. This results in more clusters, and despite an improvement in strength, the ductility falls to a very low level (2%).

The alloy of FIG. 4 illustrates that a lower Er compared to the alloy of FIG. 1 (8% Er vs. 13% Er) can achieve a comparatively “clean microstructure” and similar properties to that of alloy of FIG. 1 by combining Nd with this lower Er content.

FIG. 5 illustrates the effect of Lu, which appears to provide a similar manner to Er; however, Lu appears more tolerant to Nd additions in terms of freedom of particles and clusters compared with the alloy of FIG. 4.

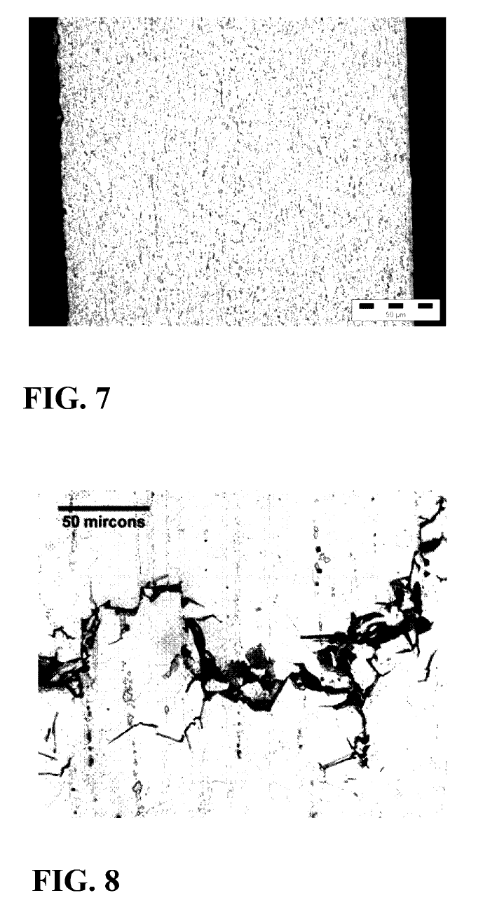

FIGS. 6 and 7 illustrate the difference in micro-structure of drawn tubes from the reference material and micro-extruded tubes from the inventive alloy MI0029. It clearly can be seen that the micro-extruded tubes have significantly less and smaller precipitates than the drawn material. In addition, the grain size of the extruded tubes is significantly reduced from ca. 15-20 μm for the as extruded bulk materials and 2-15 μm for the drawn condition.

In structural terms, it has been found that an improvement in processability and/or ductility becomes noticeable when the area percentage of particles in the alloy having an average particle size in the range of 1 to 15 μm is less than 3%, and particularly less than 1.5%. Most preferred is the area percentage of particles having an average size greater than 1 μm and less than 10 μm being less than 1.5%. These detectable particles tend to be brittle.

In structural terms, it has been found that an improvement in processability and/or ductility and/or strength becomes noticeable when the grain size is reduced.

Corrosion Behavior

The corrosion behavior of selected alloy systems was investigated in greater detail on the basis of three standardized tests. The results of these tests are summarized in Table 2 and Table 4.

Salt Fog Test

First, a standardized test to evaluate the industrial usability of the alloys was performed using a 5% NaCl-containing spray mist according to ASTM B117. The samples were exposed to the test conditions for the required number of days, and then the corrosion product was removed by boiling in a 10% chromium trioxide solution. The weight loss of the samples was determined and expressed in mpy (mils penetration per year) as is customary in international practice.

Immersion in SBF

The corrosion resistance also depends on the corrosion medium. Therefore, an additional test method has been used to determine the corrosion behavior under physiological conditions in view of the special use of the alloys.

For storage in SBF (simulated body fluid) with an ionic concentration of 142 mmol/L Na⁺, 5 mmol/L K⁺, 2.5 mmol/L Ca²⁻, 1 mmol/1 Mg²⁺, 1 mmol/1 SO₄²⁻, 1 mmol/1 HPO₄²⁻, 109 mmol/1 Cl⁻ and 27 mmol/L HCO₃⁻ cylindrical samples of the extruded material are completely immersed in the hot medium for 7 days at nominally 37° C. The corrosion product is then removed by boiling in a 10% chromium trioxide solution. As for the ASTM B117 test, the weight loss of the samples was determined and expressed in mpy.

An important factor to note is that the absolute value can vary with each batch test. This is can make comparison of absolute values difficult. To resolve this, a standard (known reference type alloy WE43 (for example MI0034 type alloy) is tested with each batch of alloys tested. The reference is then used as a basis to compare any improvements. The reference is given the value 100%, and values less than this show an improvement (less degradation).

However, the corrosion resistance also depends on the corrosion medium and the mechanical load conditions acting at the same time. Therefore, an additional test method has been used to determine the Stress Corrosion Cracking (SCC) behavior under physiological conditions in view of the special use of the alloys.

EAC/SCC in SBF

Stress tests in SBF media were carried out to identify susceptibility to Environmental Assisted Cracking (EAC), also known as stress corrosion cracking (SCC), and to compare the alloys of the invention to a WE43 type reference alloy.

The test involves testing a machined cylindrical specimen containing sharp notches to act as stress initiators. The samples were loaded with a fixed weight via a cantilever mechanism. The specimen was located inside a container which allowed SBF media to immerse the sample to a level greater than the notched portion of the sample. Media was changed every two days to minimize any compositional changes during testing. Pass criteria was at least 250 hours continuous exposure to SBF media without failure. The stress value whereby failure occurred in ≧250 hours was defined as the threshold value which is reported in Table 4.

To determine the susceptibility of the alloy tested to the SBF media, each batch was tested to failure in air. This value was compared with the threshold stress value in SBF as described above, and the reduction in failure stress expressed as a % of “notched strength in air”. It is likely that closer the value is to 100%, the less susceptible the material is to EAC.

Mg-Ion Release From Micro-Extruded Tubes and Fully Processed Stents

However, since it is also known that the thermo-mechanical treatments and the surface conditions of materials affect the corrosion behaviour, we also characterized the corrosion resistance of the materials by quantification of the Mg ion release from micro-extruded tubes and actual fully processed stents in SBF.

The samples for the Mg ion release tests were manufactured from micro-extruded tubes as described above. Furthermore, the extruded tubes were laser beam cut to the shape of stents, electro-polished, crimped on balloon catheters, sterilized and expanded into hoses of appropriate size where they were surrounded by flowing SBF. Samples from the test solution were taken at different time points and subject to quantitative Mg ion evaluation by means of an ion chromatographic procedure described elsewhere. Drawn tubes of WE43 and the respective stent served as references.

Results of Salt Fog Test

Surprisingly, the results of the salt fog test in NaCl atmosphere clearly indicate that an increasing content of Er (Ho, Lu, Tb, Tm) reduces the corrosion resistance significantly. More surprising is the oppositional corrosion behaviour of the inventive alloys in SBF, a solution simulating the actual biological service environment of vascular implants much better than the salt fog test, since previous investigations indicated a distinct correlation between mass loss in salt fog (ASTM B117) and mass loss in SBF. The SBF immersion test revealed a significant reduction of the mass loss with increasing content of Er from 6 wt % to 14 wt %. From about 18 wt % Er on, the corrosion rate deteriorates further.

Results of Immersion in SBF

Tests of the alloys of the invention immersed in SBF illustrate the reduction of the degradation rate (corrosion). This is best viewed as a % of the reference alloy. In the best case, examples from the invention show a greater than 10 fold improvement in degradation.

Generally speaking, as Er, Ho, Lu, Gd (as well as Tb and Tm) increase, the degradation resistance also improves, i.e. the measured mass loss becomes lower compared to the reference. Within the alloy additions mentioned above, there also appear to be some differences between the elements individually, with some showing better performance. It would be expected that combinations of some of the HREs could provide synergistic benefits at appropriate alloy contents.

TABLE 4

| | | | | | | | | | | | | | | | | Corro- | EAC | |
|---|---|---|---|---|---|---|---|---|---|---|---|---|---|---|---|---|---|---|
| | | | | | | | | | | | | | | | | sion | Properties | |
| | | | | | | | | | | | | | | | | Prop- | | UTS |
| | | | | | | | | | | | | | | | | erties | | in |
| | | | | | | | | | | | | | | | | in | | SBF |
| | | | | | | | | | | | | | | | | SBF | | com- |
| | | | | | | | | | | | | | | | | % of | | pared |
| | | | | | | | | | | | | | | | | WE43 | UTS | with |
| Chemical Analysis | | | | | | | | | | | | | | | | Ref- | in | UTS |
| Y | Nd | Zr | Gd | Dy | Yb | Er | Sm | La | Ce | Pr | Ho | Lu | Al | Fe | TRE ¹ | erence | SBF | in air |

| | | | | |
|---|---|---|---|---|
| ID | % by weight | alloy | MPa | % |

Reference alloy

| | | | | | | | | | | | | | | | | | |
|---|---|---|---|---|---|---|---|---|---|---|---|---|---|---|---|---|---|
| MI0047 | 4.00 | 2.2 | 0.58 | 0.44 | 0.5 | 0.00 | 0.01 | 0.00 | 0.00 | 0.00 | 0.00 | 0.01 | 0.002 | 1.0 | 100 | 243 | 60 |

Experimental Alloys

| | | | | | | | | | | | | | | | | | | | |
|---|---|---|---|---|---|---|---|---|---|---|---|---|---|---|---|---|---|---|---|
| MI0046 | 4.2 | 2.1 | 0.82 | 0.25 | 0.29 | 0.00 | 0.01 | 0.01 | 0.00 | 0.00 | 0.00 | | 8 | 0.01 | 0.002 | 8.6 | 17 | 302 | 70 |
| MI0031 | 3.90 | 0.00 | 0.74 | 0.00 | 0.02 | 0.00 | 12.69 | 0.02 | 0.00 | 0.02 | 0.00 | | | 0.01 | 0.003 | 12.8 | 14 | 285 | 65 |
| DF9546 | 4.00 | 0.00 | 0.63 | 0.00 | 0.00 | 0.00 | 6.61 | 0.00 | 0.00 | 0.00 | 0.00 | | | 0.001 | 0.002 | 6.6 | 40 | 268 | 65 |
| MI0036 | 3.60 | 0.03 | 0.83 | 0.00 | 0.00 | 0.00 | 14.00 | 0.01 | 0.00 | 0.01 | 0.00 | | | 0.01 | 0.004 | 14.0 | 8 | 285 | 60 |
| DF9561 | 4.00 | 2.20 | 0.61 | 0.00 | 0.00 | 0.00 | 7.14 | 0.02 | 0.00 | 0.00 | 0.00 | | | 0.01 | 0.003 | 7.2 | 49 | 270 | 60 |
| MI0037 | 3.90 | 0.04 | 0.80 | 0.00 | 0.00 | 0.00 | 18.00 | 0.03 | 0.00 | 0.03 | 0.00 | | | 0.01 | 0.004 | 18.1 | 25 | 169 | <45 |
| MI0045 | 3.7 | 2.1 | 0.73 | 0.43 | 0.29 | 0.00 | 0.03 | 0.06 | 0.00 | 0.03 | 0.00 | 8 | | 0.008 | 0.003 | 8.8 | 26 | <182 | <40 |

Additional Examples

| | | | | | | | | | | | | | | | | |
|---|---|---|---|---|---|---|---|---|---|---|---|---|---|---|---|---|
| DF9400 | 5.50 | 0.00 | 0.35 | 7.19 | 0.00 | 0.00 | 0.02 | 0.05 | 0.00 | — | — | 0.002 | 7.3 | 58 | 210 | 45 |

| | | | |
|---|---|---|---|
| MI0033 | AZ91 (8.5% A1, 0.6% Zn, 0.2% Mn, 0.002% Fe) | <78 | <20 |

Results of EAC/SCC in SBF

Table 4 provides data on the EAC tests. Taking a WE43 type alloy (DF9319) as a reference, it can be seen that as the HRE content increases, the absolute tolerable stress increases. This improvement is also seen as a % of the actual strength of the material when tested in air (no SBF media effect). The closer this value is to 100%, the less the fracture is related to the media and, therefore, the less prone the material is likely to be to EAC (SCC) in that media.

Er additions perform well to at least 14 wt %; however, at 18 wt % the performance is reduced to less than that of the reference WE43 type alloy. Other HREs perform in different ways; for example, only 4% Gd addition gives the best EAC resistance of the alloys tested, and Lu also appears good, but Ho performs poorly.

FIG. 8 shows the fracture appearance of alloy DF9400. The fracture shows primary and secondary cracking. This type of cracking with secondary cracking remote from the primary crack can be representative of SCC.

Discussion: Mg-Ion Release from Micro-Extruded Tubes and Fully Processed Stents

TABLE 5

| | Bulk material | Micro-extruded tube | Stent |
|---|---|---|---|
| ID | in % of respective reference | | |
| DF9546 | 79 | 52 | 12 |
| MI0029 | 47 | 29 | 49 |
| MI0023 | 42 | 29 | 9 |
| MI0012 | 39 | 62 | 13 |
| MI0026 | 20 | 13 | 12 |

Table 5 shows a comparison of the Mg ion release from the bulk material, the extruded tubes, and the respective stents from these extruded tubes. Values are given as a percentage of the respective reference material (reference WE43 bulk materials from Table 2 as reference for the inventive bulk material, drawn tube of WE43 for the extruded tubes of the inventive alloys, and stents from drawn tube of WE43 for the stent manufactured from extrude tubes of the inventive alloys).

The Mg ion release tests with micro-extruded tubes and the respective stents, as well as with the drawn tubes and the respective stents as the respective references, revealed that the inventive alloys exhibit significantly less Mg ion release (20-80% less Mg ion release than the reference material), indicating a significantly higher corrosion resistance. Furthermore, the inventive alloys become about 20% to 80% more corrosion resistant than the drawn reference material when processed by micro-extrusion. Further improvements (50-90% less Mg ion release than stents from drawn reference material) can be gained through proper electro-polishing to produce a stent. While the inventive alloys show improved corrosion properties after processing, the corrosion properties of the reference material drop during tube-drawing and polishing.

is This can be explained by the microstructure of the bulk materials (see the section “Mechanical properties and metallurgical description of the alloy”) resulting from the inventive changes of the composition and the processed material where the drawn reference tube shows significantly more precipitates than the micro-extruded material. These precipitates, which are known to be electrochemically more noble, also exist on the polished surface, creating galvanic couples that accelerate dissolution rates.

In addition, the grain size is significantly reduced from ca. 15-20 μm in the as extruded and drawn condition to 2-15 μm in the micro-extruded condition.

### Example

### Mg-4Y-2Nd-8Er-0.6Zr (M10029) & Mg-4Y-8Er-0.6Zr (DF9546)

High purity (>99.9%) magnesium ingots are smelted in steel crucibles at 500-800° C. The melt is protected from burning and sludge formation using fluxless techniques with mixtures of protective gases, e.g. CO₂/2% SF₆ or argon/2% SF_{6.} After smelting the pure magnesium ingots, the temperature is raised to 680-860° C., and the respective amounts of alloy ingredients of Y, Nd and Er and Zr are added.

Before casting in a water-cooled mold to form bars with a nominal diameter of 120 mm and a length of 300 mm, the melt is homogenized by stirring. After casting and cooling, the bars are machined to a nominal diameter of 75 mm with a length of 250 mm and homogenized for 8 hours at approximately 525° C.

The material is then reheated to 400-500° C., preferably 450° C., and extruded with the help is of a hydraulic press. The resulting round rods have a diameter of 12.7 mm. Before further processing or testing, 30 cm long pieces are removed from the start and end of the extrusions. The mechanical properties of the extruded bulk materials are:

MI0029:

YTS=246 MPa which is ca. 35 MPa higher than for WE43.

UTS=322 MPa which is ca. 30 MPa higher than for WE43.

E=18% which is ca. 8% less than for WE43.

The corresponding microstructure is depicted in FIG. 4.

DF9546:

YTS=195 MPa which is ca. 15 MPa less than for WE43.

UTS=283 MPa which is ca. 7 MPa less than for WE43.

E=24% which is 2% less than for WE43.

In particular, for medical applications as vessel scaffolds and stents in the vascular field, the extruded material must be further processed into tubes, e.g. by drawing or microextrusion. A stent is an endoluminal endoprosthesis having a carrier structure that is formed of a hollow body which is open at its ends, and the peripheral wall of which is formed by a plurality of struts connecting together which can be folded in a zig-zag or meander-shaped configuration, where the struts have typical dimensions in width and thickness of 30-450 μm.

Further processing of the extruded alloys into such above mentioned tubes is accomplished by a micro-extrusion process. For micro-extrusion, slugs are machined from the bulk material. These slugs are processed by a hot pressing process at elevated temperatures between 200° C. and 480° C. and extrusion speeds of 0.001 mm/s to 600 mm/s. Typical dimensions for micro-extruded tubes for vessel scaffolds have length of not less than 30 mm, a diameter of ca. 2 mm and a wall thickness between 50 and 400 μm.

Beside of the already described mechanical and corrosion properties, the mechanical properties of the micro-extruded tubes of these particular alloys compared to the WE43 tubes is are:

MI0029:

YTS=189 MPa which is ca. 25 MPa higher than for drawn WE43 tubes.

UTS=316 MPa which is ca. 66 MPa higher than for drawn WE43 tubes.

E=26% which is ca. 6% higher than drawn WE43 tubes.

The corresponding microstructures are given in FIG. 7.

DF9546:

YTS=173 MPa which is ca. 10 MPa higher than for drawn WE43 tubes.

UTS=261 MPa which is ca. 11 MPa higher than for drawn WE43 tubes.

E=29% which is ca. 9% higher than drawn WE43 tubes.

To evaluate the susceptibility to environmental assisted cracking, and in particular corrosion fatigue upon an in vivo like cyclic loaded vascular scaffold, we produced actual stents from the micro-extruded tube by laser cutting and electro polishing.

Prior to testing, the stents were crimped on balloon catheters to a diameter of less than 1.5 mm and sterilized, e.g. ETO (Ethylene Oxide Sterilization) or e-beam (electron beam sterilization). The stents were than over-expanded to their nominal diameter plus 0.5 mm into mock arteries with respective diameters which were previously filled with simulated body fluid (SBF). Previous tests have shown that over-expansion to about 1 mm in diameter is possible for the new alloy while the same stent manufactured from WE43 tolerates significantly less over-expansion. The improved dilatation reserve of the inventive alloys contributes significantly to device safety in clinical practice.

The mock arteries with the stent inside are placed in a test chamber where a cyclic physiological load is applied. After certain periods of time (14 and 28 days), some arteries are transferred into another test chamber where the radial strength of the stent can be measured. Some other arteries are filled with epoxy resin for metallographic determination of the remaining load bearing cross section of the stent struts. For comparison, we used the same stent design manufactured from WE43 tubing.

The results of the relative degradation score, which is defined as the percentage of metal remaining during corrosion normalized to the respective value of the reference, which are depicted in FIG. 10, impressively indicate that the inventive alloys exhibit significantly less uniform corrosion (−25%) than the reference when cyclic loaded in a corrosive environment.

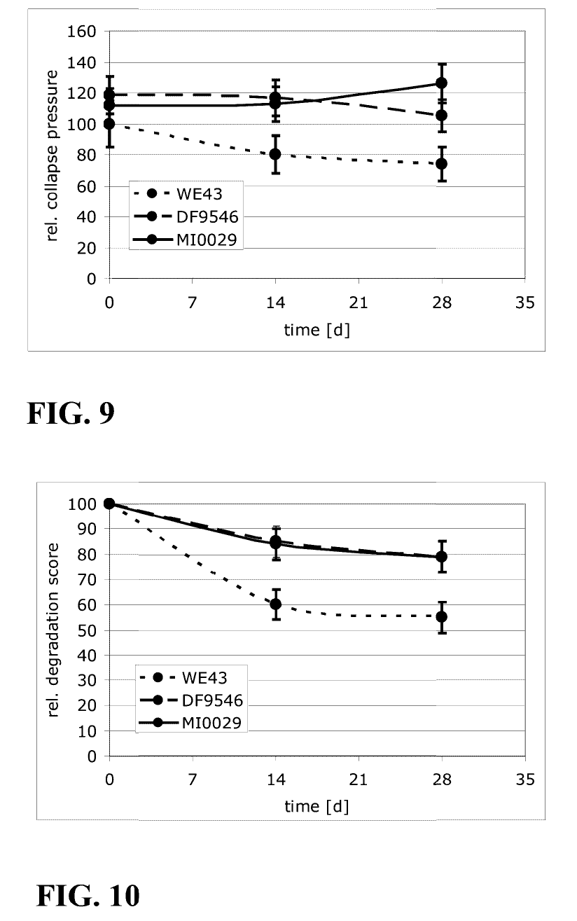

The results of the relative collapse pressure measurement, which is defined as the absolute collapse pressure normalized to the initial collapse pressure of the reference, which are depicted in FIG. 9, also impressively indicate that stents manufactured from inventive alloys exhibit a significant higher initial collapse pressures (+10%) as an result of the higher strength, the lower yield ratio, and the higher strain hardening. Furthermore, the stents maintain that high initial level of scaffold ability over a significantly longer period of time without fractures or fragmentation, indicating significantly less susceptibility to environmental assisted cracking, in particular corrosion fatigue.

The positive effect of the addition of highly soluble Er to Mg—Y—Zr and Mg—Y—Nd—Zr-alloys becomes also obvious especially when comparing the microstructure after microextrusion with the microstructure of micro-extruded WE43. In FIG. 6, it can be seen that a large portion of linear agglomerates of large precipitates (stringers) is present in the WE43 tube (FIG. 7), while the particles are significantly finer and more evenly distributed in the MI0029 tube. Those particles may act as cathodes for galvanic corrosion, as well as crack ignition sides during static or cyclic loading, and are therefore unfavourable.

1. Implant made in total or in parts of a biodegradable magnesium alloy comprising:
Y: 0-10.0% by weight
Nd: 0-4.5% by weight
Gd: 0-9.0% by weight
Dy: 0-8.0% by weight
Ho: 0-19.0% by weight
Er: 0-23.0% by weight
Lu: 0-25.0% by weight
Tm: 0-21.0% by weight
Tb: 0-21.0% by weight
Zr: 0.1-1.5% by weight
Ca: 0-2.0% by weight
Zn: 0-1.5% by weight
In: 0-12.0% by weight
Sc: 0-15.0% by weight
incidental impurities up to a total of 0.3% by weight;
wherein:
a) a total content of Ho, Er, Lu, Tb and Tm is equal or more than 5.5% by weight;
b) a total content of Y, Nd and Gd is equal or more than 2% by weight
c) a total content of all alloy compounds except magnesium is equal or more 8.5% by weight; and
d) the balance to 100% by weight being magnesium.

2. The implant of claim 1, wherein the content of Y is 1.0-6.0% by weight.

3. The implant of claim 1, wherein the content of Nd is 0.05-2.5% by weight.

4. The implant of claim 1, wherein the content of Gd is 0-4.0% by weight.

5. The implant of claim 1, wherein a total content of Y, Nd and Gd in the Mg alloy is more than 3.0% by weight.

6. The implant of claim 1, wherein the content of Dy is 0-6.0% by weight.

7. The implant of claim 1, wherein the content of Ho is 4-15.0% by weight.

8. The implant of claim 1, wherein the content of Er is 4.0-15.0% by weight.

9. The implant of claim 1, wherein the content of Lu is 4.0-15.0% by weight.

10. The implant of claim 1, wherein the content of Tm is 4.0-15.0% by weight.

11. The implant of claim 1, wherein the content of Tb is 4.0-15.0% by weight.

12. The implant of claim 1, wherein the total content of Dy, Ho, Er, Lu, Tb and Tm is in the range of 6.5-25.0% by weight.

13. The implant of claim 1, wherein the content of Zr is 0.2-0.6% by weight.

14. The implant of claim 1, wherein the content of Ca is 0-1.0% by weight.

15. The implant of claim 1, wherein the content of Zn is 0-0.5% by weight.

16. The implant of claim 1, wherein the content of In is 0-2.5% by weight.

17. The implant of claim 1, wherein the total content of In, Zr, Ca and Zn is in the range of 0.2-2.0% by weight.

18. The implant of claim 1, wherein the implant is a stent.

19. A method for forming an implant, comprising:
preparing a biodegradable magnesium alloy with the following composition:
Y: 0-10.0% by weight
Nd: 0-4.5% by weight
Gd: 0-9.0% by weight
Dy: 0-8.0% by weight
Ho: 0-19.0% by weight
Er: 0-23.0% by weight
Lu: 0-25.0% by weight
Tm: 0-21.0% by weight
Tb: 0-21.0% by weight
Zr: 0.1-1.5% by weight
Ca: 0-2.0% by weight
Zn: 0-1.5% by weight
In: 0-12.0% by weight
Sc: 0-15.0% by weight
incidental impurities up to a total of 0.3% by weight
wherein:
a) a total content of Ho, Er, Lu, Tb and Tm is equal or more than 5.5% by weight;
b) a total content of Y, Nd and Gd is equal or more than 2% by weight
c) a total content of all alloy compounds except magnesium is equal or more 8.5% by weight; and
d) the balance to 100% by weight being magnesium;
forming the implant from the biodegradable magnesium alloy.

20. The method of claim 19, wherein the implant is a stent.
